(19) United States
(12) Patent Application Publication
(10) Pub. No.: US 2025/0009255 A1
(43) Pub. Date: Jan. 9, 2025

(54) DIAGNOSTIC PLATFORM FOR TESTING EXHALED BREATH CONDENSATE

(71) Applicant: John James Daniels, Madison, CT (US)

(72) Inventor: John James Daniels, Madison, CT (US)

(21) Appl. No.: 18/443,194

(22) Filed: Feb. 15, 2024

Related U.S. Application Data
(63) Continuation of application No. PCT/US22/74961, filed on Aug. 15, 2022.
(60) Provisional application No. 63/233,473, filed on Aug. 16, 2021.

(51) Int. Cl. A61B 5/097; G01N 33/543

(52) U.S. Cl. CPC A61B 5/097; G01N 33/54366

(57) ABSTRACT

An exhaled breath condensate (EBC) collector for converting breath vapor received from the lungs and airways of a test subject into an EBC fluid biosample. The EBC collector including a cooling system for cooling a condensing surface wherein the breath vapor is coalesced into liquid droplets forming the EBC fluid sample. A biomarker concentrator concentrates a target biomarker portion in the fluid biosample to form a concentrated fluid biosample. The biomarker concentrator comprises a selectively permeable barrier for allowing excess water in the fluid biosample to pass through the selectively permeable barrier and block the target biomarker in the fluid biosample from passing through the selectively permeable barrier. A biomarker testing unit receives the concentrated fluid biosample and tests the concentrated fluid biosample for the target biomarker.

### REFERENCE TO RELATED APPLICATIONS

This is a continuation of PCT application PCT/US22/74961, filed Aug. 15, 2022, for Diagnostic Platform for Testing Exhaled Breath Condensate, which claims the benefit of priority of U.S. provisional patent application No. 63/233,473, invented by Daniels, filed on Aug. 16, 2021, for Diagnostic Platform for Testing Exhaled Breath Condensate, the teachings of all of the foregoing applications of which are incorporated by reference herein in their entirety.

### BACKGROUND OF THE INVENTION

The exemplary and non-limiting embodiments of this invention relate generally to diagnostic systems, methods, devices and computer programs and, more specifically, relate to digital and analog diagnostic devices for detecting a biomarker of a biological agent such as a coronavirus, lung cancer, tuberculosis, asthma, and other respiratory ailments and conditions, and/or blood borne biomarkers and other biomarkers that are present in the exhaled breath of a test subject.

The present invention also pertains to a device architecture, specific-use applications, and computer algorithms used to detect biometric parameters for the treatment and monitoring of physiological conditions in humans and animals.

This section is intended to provide a background or context to the exemplary embodiments of the invention as recited in the claims. The description herein may include concepts that could be pursued but are not necessarily ones that have been previously conceived, implemented or described.

Therefore, unless otherwise indicated herein, what is described in this section is not prior art to the description and claims in this application and is not admitted to being prior art by inclusion in this section.

Testing for biomarkers that indicate exposure, infection, progression and recovery from a disease condition, such as COVID-19 can be used to screen individuals for infection and help slow the spread of the virus. For example, protein and RNA testing for active virus shows who is currently contagious. Antibody testing can be used to find the members of a population that have recovered from the virus.

Diagnostics of SARS-COV-2 infection using real-time reverse-transcription polymerase chain reaction (RT-PCR) on nasopharyngeal swabs is now well-established, with saliva-based testing being lately more widely implemented for being more adapted for self-testing approaches. The procedure to obtain nasal swab samples is not only uncomfortable, but requires specialized personal with risk of contaminating the person performing the test. Saliva tests have the advantage of being simpler to perform, less invasive with limited risks and RT-PCR on saliva specimens has becoming more widely implemented. The viscose nature of saliva together with the presence of saliva proteases, responsible for the proteolytic activity of saliva, make the direct application of saliva samples challenging. It is well known that the major mechanisms of COVID-19 spread are airborne and contact infections primarily due aerosol droplets expelled from the lungs and airways of infected persons. There is therefore a growing need for sample collection by patients themselves and a simple to use testing system that can detect a target biomarker indicative of a pathogenic infection.

### SUMMARY OF THE INVENTION

The below summary section is intended to be merely exemplary and non-limiting. The foregoing and other problems are overcome, and other advantages are realized, by the use of the exemplary embodiments of this invention.

In accordance with a non-limiting exemplary embodiment, an exhaled breath condensate (EBC) collector for converting breath vapor received from the lungs and airways of a test subject into an EBC fluid biosample. The EBC collector including a cooling system for cooling a condensing surface wherein the breath vapor is coalesced into liquid droplets forming the EBC fluid sample. A biomarker concentrator concentrates a target biomarker portion in the fluid biosample to form a concentrated fluid biosample. The biomarker concentrator comprises a selectively permeable barrier for allowing excess water in the fluid biosample to pass through the selectively permeable barrier and block the target biomarker in the fluid biosample from passing through the selectively permeable barrier. A biomarker testing unit receives the concentrated fluid biosample and tests the concentrated fluid biosample for the target biomarker.

In accordance with another non-limiting exemplary embodiment, a method for concentrating a target analyte in an exhaled breath condensate (EBC) sample, comprising the steps of: collecting the EBC sample from the lungs and airways of a test subject, the EBC containing the target analyte; providing a super absorbent polymer in a flow path of the EBC where during the collection, the EBC sample is contacted with the super absorbent polymer, where the super absorbent polymer absorbs a portion of water from the EBC sample and does not absorb the target analyte resulting in a concentration of the target analyte in remaining water in the EBC sample.

In accordance with another non-limiting exemplary embodiment, a method for assembling an array of applied-field-reactive capture molecule conjugates, includes providing a dissolvable adhesive film. A carrier fluid is provided that is a non-solvent for the dissolvable adhesive film. The carrier fluid has randomly dispersed applied-field-reactive capture molecule conjugates. An aligning field is applied to the carrier fluid for assembling the applied-field-reactive capture molecule conjugates onto the dissolvable adhesive film; and evaporating the carrier fluid leaving the assembled applied-field-reactive capture molecule conjugates fixed on the dissolvable adhesive film.

In accordance with another non-limiting exemplary embodiment, a method for detecting a target analyte includes providing a capture molecule structure having a ligand end and a polarizable end. When the capture molecule structure is disposed in a carrier fluid, the capture molecule structure is a free floating element. A target analyte is provided as another free floating element in the carrier fluid. The ligand end of the capture molecule structure binds to the target analyte and forms a free floating polar conjugate having a positive end and a negative end. The polar conjugate is aligned in the carrier fluid in an electric field and an electrical property of the aligned polar conjugate is measured to detect the target analyte.

In accordance with another non-limiting exemplary embodiment, a capture molecule conjugate for detecting a target analyte, includes an applied-field-reactive capture molecule conjugate having at least one applied-field-responsive end and at least one capture molecule end. Each said capture molecule end binds to and captures the target analyte. The binding of each said capture molecule end to the target analyte increases an electrical charge difference between the at least one applied-field-responsive end and the at least one capture molecule end.

In accordance with another non-limiting exemplary embodiment, an exhaled breath condensate (EBC) collector converts breath vapor received from the lungs and airways of the test subject into a fluid biosample. The EBC collector includes a condensate-forming surface. A thermal mass is provided in thermal connection with the condensate-forming surface. The thermal mass comprises at least a first chemical reagent and a second chemical reagent combinable to form an endothermic chemical reaction for absorbing thermal energy from the condensate-forming surface for converting the exhaled breath vapor to the EBC. A fluid transfer system transfers the EBC to at least one of a testing unit and an EBC containment vessel comprising at least one of a fluid conductor, a pooling area, and a microfluidics transfer path for controlling a flow of the fluid biosample received from the EBC collector. A target biomarker releasing material is disposed in said at least one of the fluid conductor, pooling area and microfluidics transfer path.

In accordance with another non-limiting exemplary embodiment, a method for forming a condensate collector having fluid conductor channels on a substrate for guiding a flow of fluid towards a desired direction includes providing the substrate having a surface having a relatively lower energy surface property. A textured structure forms the fluid conductor channels on the surface having a relatively higher energy surface property for guiding a flow of fluid towards a desired direction.

### BRIEF DESCRIPTION OF THE DRAWINGS

To easily identify the discussion of any particular element or act, the most significant digit or digits in a reference number refer to the figure number in which that element is first introduced.

FIG. 1 shows a face mask and schematically illustrates an Exhaled Breath Condensate (EBC) collector, fluid transfer system, biosensor testing system and near field communication (NFC) antenna and signal condition circuit all disposed within the confined volume of the inside of the face mask.

FIG. 2 shows the inside of the mask splayed open with components for collecting and testing EBC and exhaled breath aerosols (EBA).

FIG. 3 is a block diagram of one possible and non-limiting exemplary system in which some of the exemplary embodiments may be practiced.

FIG. 4 is a logic flow diagram for Applied Probabilistic Analysis to determine the detecting of a target biomarker, and illustrates the operation of an exemplary method, a result of execution of computer program instructions embodied on a computer readable Memory, functions performed by logic implemented in hardware, and/or interconnected means for performing functions in accordance with exemplary embodiments.

FIG. 5 is a logic flow diagram for Data Acquisition and Transmission for Trusted Receiver and Population Study use-cases, and illustrates the operation of an exemplary method, a result of execution of computer program instructions embodied on a computer readable Memory, functions performed by logic implemented in hardware, and/or interconnected means for performing functions in accordance with exemplary embodiments.

FIG. 6 is a block diagram of the basic components for testing EBC and transmitting the test result to a smartphone and/or cloud server.

FIG. 7 shows a KN95 face mask with a retrofittable testing system including an EBC collector having an aluminum foil condensate-forming surface, embossed fluid conductor channels, a fluid transfer system and electronic biosensor electrodes.

FIG. 8 shows the testing system retrofit into the KN95 mask.

FIG. 9 shows the KN95 mask having removable testing and communication electronics disposed on the outside of the mask.

FIG. 10 shows an Exhaled Breath Condensate Collector and a testing area with ganged syndromic testing biosensors.

FIG. 11 shows a pooling area for immersing the biosensors in collected EBC.

FIG. 12 shows an assembled EBC collector and pooling area.

FIG. 13 shows the sizes of an engineered capture molecule, an S-protein, a virus particle and a water molecule.

FIG. 14 shows an EBC concentrator having a semipermeable membrane for separating excess water from EBC with concentrated virus particles.

FIG. 15 shows the addition of a super absorbent polymer in the EBC concentrator.

FIG. 16 shows the addition of a surfactant or lysing agent in the EBC concentrator.

FIG. 17 shows an EBC droplet having concentrated virus particles and lysed proteins.

FIG. 18 is a cross section showing an EBC concentrator with a semipermeable membrane and wick disposed adjacent to a thermal mass.

FIG. 19 is a cross section showing an EBC concentrator with a fluid conductor with lysing material and having a semipermeable membrane and wick in an EBC pooling area.

FIG. 20 shows endothermic reaction constituents.

FIG. 21 shows a retrofittable endothermic EBC collector and a pre-existing face mask.

FIG. 22 shows an endothermic EBC collector having hydrophilic channels on a hydrophobic field.

FIG. 23 shows an endothermic EBC collector retrofit into an existing face mask.

FIG. 24 shows a reacted endothermic EBC collector and collected EBC droplets.

FIG. 25 shows an endothermic EBC collector with a pooling area and dry buffer/surfactant.

FIG. 26 shows an Exhaled Breath Condensate Collector and a testing area with ganged syndromic testing biosensors.

FIG. 27 shows a pooling area for immersing the biosensors in collected EBC, with dry buffer/surfactant in the pooling area.

FIG. 28 shows an assembled EBC collector and pooling area with dry buffer/surfactant in the pooling area.

FIG. 29 shows a Teflon condensing surface with a textured fluid conductor.

FIG. 30 is a photomicrograph showing tunable water adhesion structures fabricated by laser ablation.

FIG. 31 shows a roll-to-roll process for mass producing a condensing surface with a textured fluid conductor.

FIG. 32 shows a sheet of condensing surfaces with textured fluid conductors.

FIG. 33 shows a capture molecule structure having a label attached to a ligand.

FIG. 34 shows a capture molecule structure having a polarizable end and a ligand end for capturing a target analyte.

FIG. 35 shows the capture molecule structure with a captured target analyte forming a polar conjugate.

FIG. 36 shows the alignment and testing of the polar conjugate to detect the target analyte in a fluid sample.

FIG. 37 shows a carrier fluid comprising a fluid sample with free floating target analyte molecules mixed with capture molecule structures to form free-floating polar conjugates in the fluid sample.

FIG. 38 shows the free-floating polar conjugates aligning in an applied electric field.

FIG. 39 shows the free-floating polar conjugates aligned in the applied electric field.

FIG. 40 shows a proof-of-concept laboratory engineered mask and EBC collection results.

### DETAILED DESCRIPTION

Below are provided further descriptions of various non-limiting, exemplary embodiments. The exemplary embodiments of the invention, such as those described immediately below, may be implemented, practiced or utilized in any combination (e.g., any combination that is suitable, practicable and/or feasible) and are not limited only to those combinations described herein and/or included in the appended claims.

The word “exemplary” is used herein to mean “serving as an example, instance, or illustration.” Any embodiment described herein as “exemplary” is not necessarily to be construed as preferred or advantageous over other embodiments. In any case, all of the embodiments described in this Detailed Description are exemplary embodiments provided to enable persons skilled in the art to make or use the invention and not to limit the scope of the invention which is defined by the claims.

Many configurations, embodiments, methods of manufacture, algorithms, electronic circuits, microprocessors, memory and computer software product combinations, networking strategies, database structures and uses, and other aspects are disclosed herein for a diagnostic or testing platform, devices, methods and systems that have a number of medical and non-medical uses.

Although embodiments are described herein for detection of biomarkers of SARS-CoV-2 virus, the systems, methods and apparatus described are not limited to any particular virus or disease, or just limited to biological use-cases. In most instances, where the term virus or COVID-19 is used, any other health or fitness related biomarker could be used instead. The description here and the drawings and claims are therefore not intended to be limited in any way to virus detection, the inventions described and claimed can be used for many diseases including lung cancer, diabetes, asthma, tuberculosis, environmental exposures, glucose, lactate, blood borne diseases and other ailments or indications of the health of the test subject. Further, the electronic biosensor, test systems, uses and methods of manufacturing described herein are not limited to the use of exhaled breath condensate. Wastewater, potable water, environmental quality samples, ambient samples and any bodily fluid can be used as the test sample. The use of aptamers and engineered capture molecules, in particular, make the inventive sensor widely useful because of the nature of selected aptamers being adaptable by specific engineering design and selection to have a binding affinity that is tailored to a corresponding target analyte. Therefore, the descriptions of innovations are not intended to be limited to a particular use-case, capture molecule, biomarker or analyte.

In immunochromatography, a capture molecule, which may be, for example, an aptamer, naturally occurring antibody, or engineered antibody, is disposed onto a surface of a porous membrane, and a sample passes along the membrane. As described herein, the term antibody, aptamer, engineered antibody, or capture molecule is used interchangeably. In some instances, a specific type of capture molecule may be described. Biomarkers in the sample is bound by the capture molecule which is coupled to a detector reagent. As the sample passes through the area where the capture molecule is disposed, a biomarker detector reagent complex is trapped, and a color develops that is proportional to the concentration or amount biomarker present in the sample.

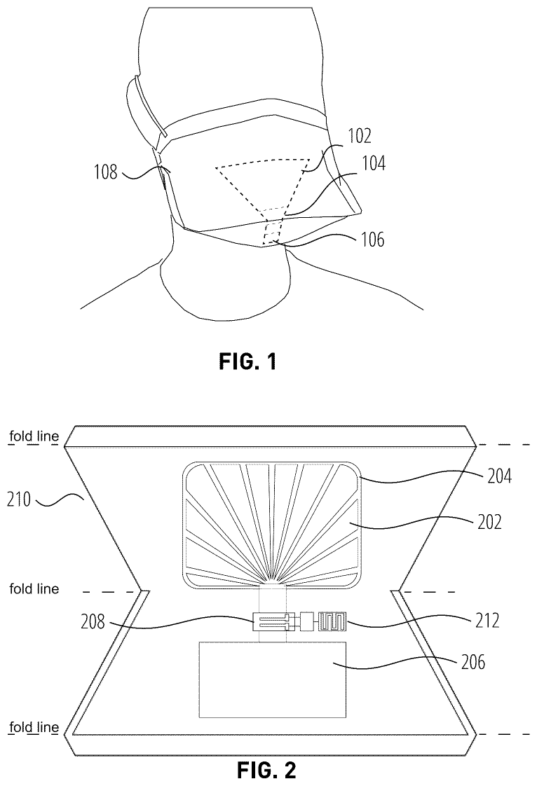

FIG. 1 shows a system including an EBC collector 102, a fluid transfer 104, an NFC antenna 106, and a face mask 108.

A face mask and a mask-based diagnostic platform is shown with an Exhaled Breath Condensate (EBC) collector, fluid transfer system, biosensor testing system and near field communication (NFC) antenna and signal condition circuit all disposed within the confined volume of the inside of the face mask. The biosensor shown is an electrical or electrochemical biosensor, such as a g-FET or other electronic biosensor construction. Alternatively, the exemplary embodiments disclosed herein can be used with other testing system, such as Lateral Flow Assays (LFAs), cellulose-based biosensors, color change reagent solutions, and the like. In some cases, the EBC collector and face mask are used to collect a fluid biosample which is tested outside the mask on a desktop or laboratory testing system (such as conventional PCR testing), in other cases, the testing systems are incorporated directly into and/or onto the face mask as a self-contained testing apparatus. In most cases, the requirements of the test subject are simply to put the face mask on and breathe normally while the biosample is obtained, and typically there is no need for a technician or other trained personnel for the test or sample collection to be completed.

In accordance with a non-limiting embodiment, a mask-based diagnostic platform is provided for detecting a biomarker received from lungs and airways of a test subject. An EBC collector is disposed on an inside of a face mask worn by the test subject. The EBC collector converts breath vapor received from the lungs and airways of the test subject into a fluid biosample. The EBC collector has a thermal mass or other cooling system, such as a frozen water/super absorbent polymer gel, a chilled metal plate or an endothermic reaction (described herein) that cools the condensing surface that receives the breath vapor at a temperature greater than a surface temperature of the condensing surface. The breath vapor is coalesced into liquid droplets on the condensing surface. The EBC collector may include a droplet harvesting structure including a field for receiving the breath vapor and forming fluid droplets, and channels for receiving the fluid droplets from the field and channeling the fluid droplets in the form of a collected fluid biosample to a fluid transfer system, such as microfluidic, capillary or other fluid conducting structure.

A biosensor is fixed to the face mask for receiving the fluid biosample from the EBC collector and testing the fluid biosample for a target biomarker. The biosensor generates a test signal dependent on at least the presence and absence of the target biomarker in the fluid biosample. A signal condition circuit, such as an amplifier, can be provided if needed to receive the test signal that is transmitted via a near field communication (NFC) antenna to a wireless receiver, such as a cellphone. Also, the signal conditioning circuit may include energy harvesting electronics that receive radio frequency energy, for example, transmitted from the cellphone and received by the NFC antenna. The energy harvesting antenna may include, for an example, a capacitor or other circuit elements so that the biosensor is operated using the harvested energy with no need for an onboard battery or other energy source.

FIG. 2 shows a system comprising an NFC antenna 106, a face mask 108, a hydrophobic surface 202, a hydrophilic structure 204, a wick 206, and a biosensor 208.

The inside of the mask-based diagnostic platform is shown splayed open with components for collecting and testing EBC. The EBC collector and testing system can be retrofitted into an existing mask or integrated into the formation of a mask. FIG. 2 shows a simple, low cost, disposable mask construction. The mask base material can be multi-stack, N95-type mask material, filter material, cloth or paper, or a breathable non-woven polymer material with micropores that allow air exchange. The EBC collector with hydrophobic fields and hydrophilic channels is fixed on the mask material. The fluid sample collected by the EBC collector is transferred by microfluidic transfer materials to the biosensor and, as described herein, can be allowed to pool on the biosensor or flow over the biosensor using a wicking material located downstream from the biosensor testing area. The biosensor testing area is small, typically a few millimeters squared or less in surface area, although a larger area and multiple biosensors or testing zones can be provided. The biosensor device has electrodes with leads that enable electrical communication with the signal conditioning circuit and NFC antenna.

This configuration creates a low cost biosample testing and communications system that can be used, along with an APP running on a cellphone to determine from the test signal a wirelessly transmittable test result depending on detecting or not detecting the target biomarker. The test result can be transmitted from the cellphone to a remote receiver, such as a cloud-based or local server. Other wired or wireless communication systems can be used. In the case of a relatively more expensive test reader and communication electronics, preferably, the electronics and battery are disposed on the outside of the mask when in use, and the EBC collector, microfluidic transfer materials, biosensor and wicking materials are disposed on the inside of the mask. After use, the electronics can be removed from the outside of the mask and sanitized for a next use. The disposable mask and components located inside the mask (and exposed to the most potential contamination) can be sealed in a suitable bag and thrown away.

To capture aerosol droplets and particulate, optionally, a dissolvable adhesive patch can also be provided on the inside surface of the mask. The mask-based diagnostic platform can be provided with this particulate capturing structure for receiving and capturing exhaled breath aerosol (EBA) particulate from airway linings of a user. The particulate capturing structure includes a dissolvable EBA sample collector film for capturing EBA particulate. In this case, for example, a forced cough while wearing the mask ejects aerosol droplets and particulate from the lungs and airways that get captured on the dissolvable adhesive patch. The fluid biosample testing components described herein can be used to provide a rapid screening test, and if a positive infection is detected, the mask can be placed into a hermetically sealed envelope and brought to a lab for more rigorous testing of the fluid biosample, aerosol droplets and particulate held by the mask and sealed in the envelope. Also, a vessel for holding the fluid biosample may be provided, or the pooling area can include a pressure sensitive adhesive strip for sealing in the collected EBC for transport to a testing lab.

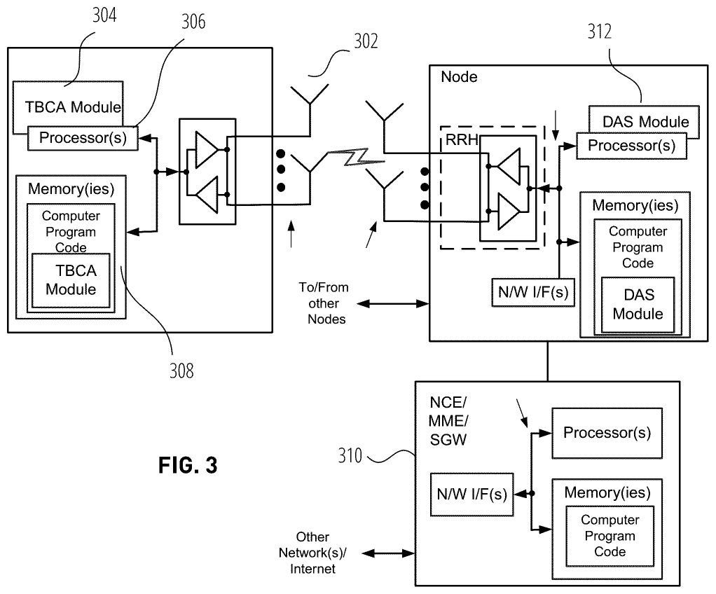

FIG. 3 shows a system comprising an antenna 302, a TBCA Module 304, a Processor 306, a Memory 308, an MME/SGW 310, and a DAS module 312.

A block diagram is shown of one possible and non-limiting exemplary system in which the exemplary embodiments may be practiced where the installed wireless communications networks and Internet can be utilized for an ultra-large scale deployment of testing for a target biomarker, such as a virus spreading through a population. Co-ordinated testing and data collection of a population can be accomplished throughout a city, state, country or even world-wide. For example, the interconnected cellular, LANs, WANs and Internet can be employed to quickly obtain important data indicating the presence of a pathogen, such an endemic or pandemic virus, at an airport, in a small community, a large city or a larger population. The test result data from many diagnostic tests can be automatically and conveniently collected and aggregated into large data sets for Big Data analysis by Machine Learning and Artificial Intelligence agents, enabling rapid pattern recognition and predictive models to be generated to indicate to governments, hospital administrators, NGOs, and other authorities where an outbreak of a virus is occurring, how rapidly the outbreak is spreading, etc. In this exemplary embodiment, the communications infrastructure of a conventional cellular communication system is utilized to quickly obtain test results from a large number of test subjects and transmit the test results in at least two data streams: 1) with patient identifying information to a trusted receiver (e.g., a patient's doctor so that the individual patient can be appropriately monitored and treated), and 2) without patient identifying information (for patient privacy purposes) for aggregation and Big Data population analysis.

A biomarker testing system (BTS) is in wireless communication with a wireless network. A BTS is a wireless biomarker testing system that can access a wireless network, such as the communications-enabled mask-based diagnostic platform described herein. The BTS includes one or more Processors, one or more Memories, and one or more Transceivers interconnected through one or more Buses 127. Each of the one or more Transceivers includes a receiver, Rx, and a transmitter, Tx. For some use-cases, there may be no need for an onboard receiver in the mask-based diagnostic unit. The one or more Buses may be address, data, or control Buses, and may include any interconnection mechanism, such as a series of lines on a motherboard or integrated circuit, or other communication equipment, and the like. The one or more Transceivers are connected to one or more antennas. The one or more Memories include computer program code. The BTS 110 includes a Target Biomarker Collection and Analysis (TBCA) module, comprising the inventive biosensor testing system described herein. An embodiment of the TBCA also includes wireless communication capabilities comprising one of or both parts, which may be implemented in a number of ways. The TBCA Module may be implemented in hardware as TBCA Module such as being implemented as part of a mask-based diagnostic system that includes the one or more Processors. The Processors of the TBCA Module may be implemented also as an integrated circuit or through other hardware such as a programmable gate array. In another example, the TBCA Module may be implemented as TBCA Module, which is implemented as computer program code and is executed by the one or more Processors, where test results are obtained in the form of analog or digital electrical signals generated by a biosensor used to test a sample, such as an EBC sample. For instance, the one or more Memories and the computer program code may be configured to, with the one or more Processors, cause the biomarker testing system to perform one or more of the operations as described herein. The BTS communicates with Node via a wireless link.

In an exemplary cellular communication model, a near field communication system is used to obtain test results from the biosensor of a mask-based diagnostic system. The NFC system enables convenient and low-cost obtainment of the test result using a conventional hand-held cellular telephone, computer, communications pad, or a dedicated wireless communication reader/transmitter/receiver.

The test results are transmitted to the Node, which is typically a base station of a wireless communications network (e.g., 5G, 4G, LTE, long term evolution or any other cellular, internet and/or wireless network communication system) that provides access by wireless devices such as a cellular telephone BTS to the wireless network. For example, at a stadium or airport, the wireless communication infrastructure of the venue can be utilized for direct communication with the mask-based diagnostic system, and/or the user's cellphone can be used as a relay.

The Node includes one or more Processors, one or more Memories, one or more network interfaces (N/W I/F(s)), and one or more Transceivers interconnected through one or more Buses. Each of the one or more Transceivers includes a receiver, Rx and a transmitter, Tx. The one or more Transceivers are connected to one or more antennas. The one or more Memories include computer program code. The Node includes a Data Acquisition and Storage (DAS) module, comprising one of or more parts, which may be implemented in a number of ways. The DAS module may be implemented in hardware as DAS module such as being implemented as part of the one or more Processors. The DAS module may be implemented also as an integrated circuit or through other hardware such as a programmable gate array. In another example, the DAS module may be implemented as DAS module which is implemented as computer program code and is executed by the one or more Processors. For instance, the one or more Memories and the computer program code are configured to, with the one or more Processors, cause the Node to perform one or more of the operations as described herein. The one or more network interfaces 161 communicate over a network such as via the links. Two or more Nodes communicate using, e.g., link. The link may be wired or wireless or both and may implement, e.g., an X2 interface.

The one or more Buses may be address, data, or control Buses, and may include any interconnection mechanism, such as a series of lines on a motherboard or integrated circuit, fiber optics or other optical communication equipment, wireless channels, and the like. For example, the one or more Transceivers may be implemented as a remote radio head (RRH), with the other elements of the Node being physically in a different location from the RRH, and the one or more Buses could be implemented in part as fiber optic cable to connect the other elements of the Node to the RRH.

The wireless network 100 may include a network control element (NCE) that may include MME (Mobility Management Entity)/SGW (Serving Gateway) functionality, and which provides connectivity with a further network, such as a telephone network and/or a data communications network (e.g., the Internet). The Node is coupled via a link to the NCE. The link may be implemented as, e.g., an S1 interface. The NCE includes one or more Processors, one or more Memories, and one or more network interfaces (N/W I/F(s)), interconnected through one or more Buses. The one or more Memories include computer program code. The one or more Memories and the computer program code are configured to, with the one or more Processors, cause the NCE to perform one or more operations.

The wireless network 100 may implement network virtualization, which is the process of combining hardware and software network resources and network functionality into a single, software-based administrative entity, a virtual network. Network virtualization involves platform virtualization, often combined with resource virtualization. Network virtualization is categorized as either external, combining many networks, or parts of networks, into a virtual unit, or internal, providing network-like functionality to software containers on a single system. Note that the virtualized entities that result from the network virtualization are still implemented, at some level, using hardware such as Processors and Memories, and also such virtualized entities create technical effects.

The computer readable Memories may be of any type suitable to the local technical environment and may be implemented using any suitable data storage technology, such as semiconductor-based Memory devices, flash Memory, magnetic Memory devices and systems, optical Memory devices and systems, fixed Memory and removable Memory. The computer readable Memories may be means for performing storage functions. The Processors may be of any type suitable to the local technical environment, and may include one or more of general purpose computers, special purpose computers, microprocessors, digital signal Processors (DSPs) and Processors based on a multi-core Processor architecture, as non-limiting examples. The Processors may be means for performing functions, such as controlling the Covid-19 Biomarker Testing System (C19TS), Node, and other functions as described herein.

In general, the various embodiments of the biomarker testing system 110 can include, but are not limited to, wireless communication components used for Bluetooth, cellular telephones such as smart phones, tablets, personal digital assistants (PDAs) having wireless communication capabilities, portable computers having wireless communication capabilities, image capture devices such as digital cameras having wireless communication capabilities, gaming devices having wireless communication capabilities, music storage and playback appliances having wireless communication capabilities, Internet appliances permitting wireless Internet access and browsing, tablets with wireless communication capabilities, as well as portable units or terminals that incorporate combinations of such functions.

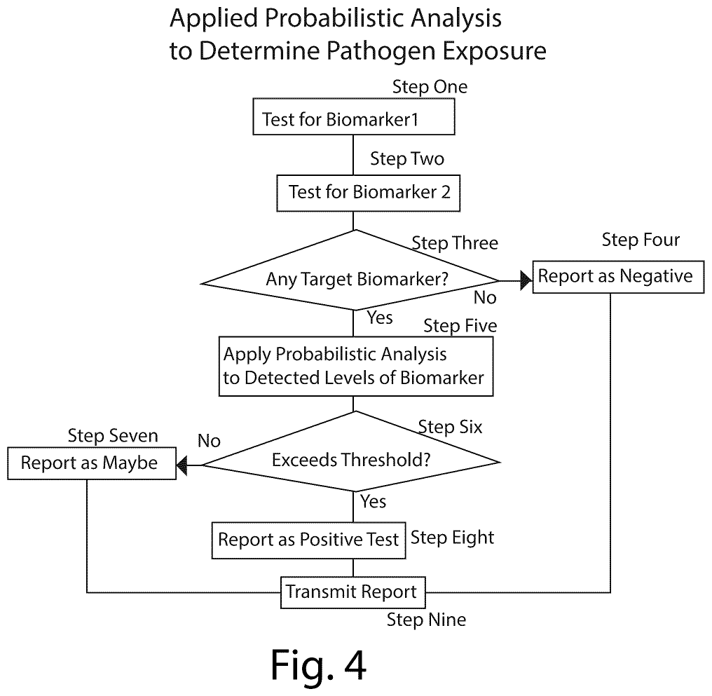

FIG. 4 is a logic flow diagram for Applied Probabilistic Analysis to determine the detection of a target biomarker, and illustrates the operation of an exemplary method, a result of execution of computer program instructions embodied on a computer readable Memory, functions performed by logic implemented in hardware, and/or interconnected means for performing functions in accordance with exemplary embodiments. For instance, the TBCA Module 140 may include multiples ones of circuit elements for implementing the functions shown in the blocks in FIG. 3, where each included block is an interconnected means for performing the function in the block. At least some of the blocks in FIG. 3 are assumed to be performed by the BTS e.g., under control of the TBCA Module at least in part.

Currently, the world is dealing with a pandemic caused by a novel coronavirus, SARS-CoV-2. This virus pathogen has caused millions of deaths, the shutting down of the economics of many nations, and trillions of dollars of economic loss world-wide. To combat the spread of the virus, billions of people around the world have halted their usual employment, entertainment and socializing activities. Testing for biomarkers that indicate exposure, infection and recovery from the virus pathogen can be used to enable a safer and more efficient restart of economic activities, while minimizing the spread of the virus, and keep a watch out for the progression of variants of the virus within a community, nation or world-wide.

For example, protein and RNA testing for active virus shows who is currently contagious. Antibody testing can be used to find the members of a population that have recovered from the virus and now may be immune to reinfection. During this current and a future pandemic, this knowledge can enable precision social distancing and more effective contact tracing, with the re-employment of a growing workforce of protected individuals and consumers. Those who remain at-risk of infection and transmission can be kept sequestered until a vaccine or other solution such as a high success rate pharmaceutical therapy is developed and made widely available. In the future, a rapid deployment of testing system, such as the mask-based diagnostic platform described herein, especially when the wireless communications infrastructure is used as described in FIGS. 3-6 and elsewhere herein, can quickly ascertain the location and speed of a pathogen spread through a population and identify those who are infected prior to traveling and bringing the virus to uninfected parts of the world.

The COVID-19 pandemic is an example of a viral pathogen that has required every nation to deploy massive resources in combating the disease, and the shutting down of economies, in an attempt to limit the spread of the virus while balancing the economic and social impacts on the population. Testing the population for the virus, tracking the pandemic spread and estimating the size of the infected population is a crucial tool to combat the current and any future endemic or pandemic outbreak.

Syndromic testing simultaneously tests for multiple pathogens with overlapping symptoms. In accordance with an exemplary embodiment, multiple biosensors are used to enable the simultaneous detection of a number of biomarkers, increasing the accuracy of test.

Stochastic analysis of data is used to handle changes that involve both randomness and uncertainty, aspects that are particularly difficult to manage during a fast spreading virus outbreak. More specifically, partially-observable stochastic analysis can account for incomplete knowledge of a contagious pathogen spreading through a population. For example, partial observation provides insights but only with a certain degree of certainty. In the case of a biological invasion or virus spread, testing for infection can often result in a false negative, so that the infection can then only be detected within a certain probability for an infected individual.

Applied probabilistic analysis can be used to improve the predictive model of an individual's infection status and in the aggregate, help to refine the testing results thresholds for an objective quantitative or qualitative testing system. In accordance with an exemplary embodiment, for the applied probabilistic analysis to determine pathogen exposure, Biomarker1 is first tested for (step one), Biomarker2 is then tested for (step two). Additionally, BiomarkerN is tested for where N can be any number of multiple biomarkers tested using the inventive testing system. If no target biomarker is detected (step three) then a Negative Test report is generated (step four). If any target biomarker is detected (step three) then probabilistic analysis may be performed depending simply on the detected presence (yes/no) or quantitative analysis (e.g., concentration) of the one or more detected biomarkers (step five). The probabilistic analysis can be performed using an updated probability model where probabilistic multipliers for the tested-for biomarkers are determined for a population. As an example, if a virus outbreak occurs earlier in time in a region or country different from the location of the currently applied testing, the probabilistic multipliers for the tested-for biomarkers can be determined from confirmed cases occurring during the earlier outbreak. A threshold can be determined for the results of the probabilistic analysis based on the probabilistic multipliers obtained from the confirmed cases, and help to improve the accuracy of the testing system. As an example, in an electronic biosensor, a threshold voltage for considering a test result as positive can be adjusted based on the probabilistic analysis of previously tested and confirmed positive cases. Over time, the accuracy and confidence of positive and negative determinations is improved based on the history of confirmed cases and obtained threshold voltages. As the database of tested cases grows, the overall testing regimen with interconnected communication, sharing and analysis of tests results is used to automatically improve the accuracy and confidence of future tests.

For examples of probabilistic analysis used for modeling the SARS-COV-2 pandemic, see, Modeling the dynamics of the COVID-19 population in Australia: A probabilistic analysis, Eshragh et al., Oct. 2, 2020, https://doi.org/10.1371/journal.pone.0240153; Estimated Incidence of Coronavirus Disease 2019 (COVID-19) Illness and Hospitalization—United States, February-September 2020 Reese, et al., CID 2021:72, June 2021; and Güemes, A., Ray, S., Aboumerhi, K. et al. A syndromic surveillance tool to detect anomalous clusters of COVID-19 symptoms in the United States. Sci Rep 11, 4660 (2021). https://doi.org/10.1038/s41598-021-84145-5, the disclosures of which are all incorporated herein in their entireties.

If the probabilistic analysis does not exceed a threshold (step six) (e.g., low concentration of a particular target biomarker, or the presence of just one weak biomarker indicating likely infection), then a Maybe Test report is generated (step seven). If the probabilistic analysis does exceed a threshold (step six) (e.g., high concentration of a particular target biomarker, or the presence of two or more biomarkers indicating likely infection), then a Positive Test report is generated (step eight). The Test Report is then transmitted (step nine) (e.g., in a manner described herein or other suitable transmission mechanism including verbal, digital, written or other communication transmission that adds to the accumulated database of test results).

The logic flow of FIG. 4 is implemented by a non-limiting embodiment of an apparatus, comprising at least one Processor; and at least one Memory including computer program code, the at least one Memory and the computer program code configured to, with the at least one Processor, cause the apparatus to perform at least the following: detecting one or more biometric parameters using a droplet harvesting structure for converting breath vapor to a fluid droplet for forming a fluid sample and a testing system having a biomarker testing zone for receiving the fluid sample and detecting the biometric parameter, where the biometric parameters are biomarkers dependent on at least one physiological change to a patient in response to a concerning condition such as a virus infection; receiving the one or more biometric parameters and applying probabilistic analysis to determine if at least one physiological change threshold has been exceeded dependent on the probabilistic analysis of the one or more biometric parameters; and activating an action depending on the determined exceeded said at least one physiological change.

In accordance with an embodiment, a biosensor testing device is provided having one or more biometric detectors each for detecting biomarkers as one or more biometric parameters. The biometric parameters are dependent on at least one physiological change to a patient or test subject, such as the production of immune response chemicals, the presence in the body of an active or deactivated virus or virus component, antibodies, antigens, virus RNA or DNA, or other biomarker inducing change (including an immune response or viral load count). A microprocessor receives the one or more biometric parameters and determines if at least one physiological change threshold has been exceeded depending on the one or more biometric parameters. An activation circuit activates an action depending on the determined physiological change. The action includes at least one of transmitting an alert, modifying a therapeutic treatment, and transmitting data dependent on at least one physiological change, the one or more biometric parameters, and therapeutic treatment.

The inventive mask-based diagnostic platform, and/or components of the platform described herein, can also be used to monitor the progression of a disease in a patient, for example, a hospitalized patient that is going through the disease progression of Covid-19. The at least one physiological change can also be in response to an applied therapeutic treatment that causes a change in the condition of the patient to enable the monitoring of the body's response to an applied therapeutic. The action can include transmitting an alert, modifying a therapeutic treatment, and transmitting data dependent on at least one of the at least one physiological change, the one or more biometric parameters, and therapeutic treatment. The microprocessor can analyze the one or more biometric parameters using probabilistic analysis comprising determining from a data set of the one or more biometric parameters whether the data set is acceptable for deciding that the at least one physiological change threshold has been exceeded. The probabilistic analysis can further comprise applying a statistical weighting to each of the one or more biometric parameters, where the statistical weighting is dependent on a predetermined value of a ranking of importance in detecting each of the at least one physiological change for said each of the one or more biometric parameters relative to others of the one or more biometric parameters.

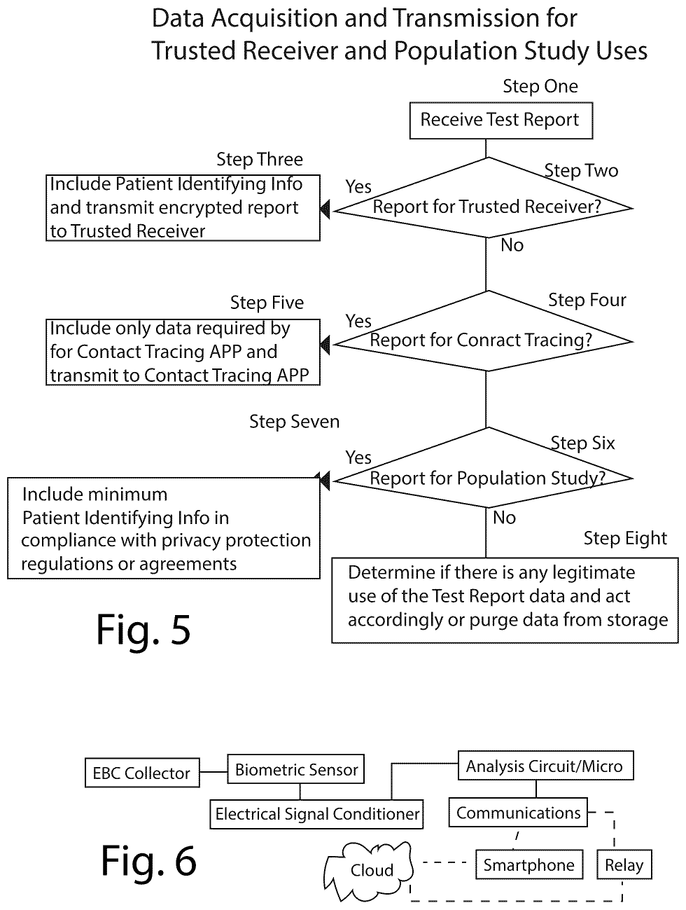

FIG. 5 is a logic flow diagram for Data Acquisition and Transmission for Trusted Receiver and Population Study uses, and illustrates the operation of an exemplary method, a result of execution of computer program instructions embodied on a computer readable Memory, functions performed by logic implemented in hardware, and/or interconnected means for performing functions in accordance with exemplary embodiments

The performance of the Data Acquisition and Transmission for Trusted Receiver and Population Study Uses process flow can be done at the testing system, Node, Smartphone, or combination of components located with the test subject or remote from the test subject, for example at storage location(s) of the acquired data. The acquired data can include patient or subject identifying information ranging from name, GPS location, list of known previous or future contacts (pre and/or post infection), prior medical history, demographics, etc. The Data Acquisition and Transmission for Trusted Receiver and Population Study Uses can be done at a secure server located anywhere on the network. For instance, the DAS module 150 may include multiple ones of circuit elements for implementing the functions shown in the blocks in FIG. 3, where each included block is an interconnected means for performing the function in the block. At least some of the blocks in FIG. 3 are assumed to be performed by a base station such as Node 170, e.g., under control of the DAS module 150 at least in part. A blockchain or other data security, storage and distribution technology can be used to enhance the privacy and controlled access to the de-identified patient data, while making this data available for researchers and authorities anywhere in the world.

The digital testing system architecture, manufacturing methods, and applications, can be used for capturing biometric data from the exhaled breath of a test subject or patient. Biometric data can be captured and transmitted continuously or at selected times with data access provided directly to a care-provider, enabling early diagnosis and ongoing monitoring, and to a researcher to gain valuable insights and assistance through AI analysis. This data detection is direct from the exhaled breath and can be provided through a wireless connection for Blockchain and AI database collection, access and analysis.

As shown in FIG. 5, a Test Report is received (step one) (e.g., from a Smartphone transmission from the patient or test subject). If the report is intended to be sent to a trusted receiver (step two), such as a patient's healthcare provider or insurance company, then an encrypted report can be generated (step three) and transmitted to the trusted receiver that includes patient identifying information. Two step verification or better, such as the verification protocols used to ensure online banking, can be used to make sure that the receiver of the patient's data is indeed a trusted receiver. If the report is not for a trusted receiver (step two) but instead is to be used for Contact Tracing (step four), then only the data required for Contact Tracking is transmitted. Also, for contact tracing, a Contact Tracing APP can be employed (step five). The Contact Tracing APP may be, for example, a system provided for identifying and notifying people who have come in contact with the test subject or patient within a given time prior or since testing for one or more target biomarkers. If the report is not for a trusted receiver (step two) or for contact tracing (step four) but instead is to be used for a Population Study (step six), then only the minimum patient identifying information in compliance with privacy regulations and/or agreements is transmitted and/or stored along with the received test report (step seven). If the report is not for a trusted receiver, contact tracing or population study (steps two, four, six) then it is determined if there is any legitimate use of the test report data and an action is taken accordingly or the data is automatically purged from storage (step eight).

FIG. 6 is a block diagram of the basic components for testing EBC and transmitting the test result to a smartphone and/or cloud server. The EBC collector provides a fluid sample that is received by the biometric detector or biosensor. An electrical signal conditioner, such as a signal amplifier, filter, etc. can be provided to condition the raw test signal from the biosensor before a microprocessor or analysis circuit determines the test result signal. After processing the conditioned signal, a test result signal is transmitted via a communications circuit, which may be the NFC system described earlier, or Bluetooth or other wired or wireless communications method and structures. A smartphone or access point relay can be used to receive the wireless test result signal and transmit it to the cloud.

In accordance with an embodiment, the electronic circuit comprises an amplification circuit for receiving the test signal from the biosensor and amplifying the test signal to an amplified electrical signal. A comparator circuit compares the amplified electrical signal with a pre-determined value based on at least one of a computer model-derived and empirically-derived electrical signal calibration of the biosensor. The calibration can be determined using at least one of a known presence and a known concentration of the target analyte in a calibration sample. The comparator circuit generates the test result signal based on the amplified electrical signal compared with the pre-determined value.

The electronic circuit can also comprise an analyte concentration circuit for determining a concentration value of the target analyte depending on the amplified electrical signal. In this case, the amplified electrical signal changes value depending on a number of target analyte molecules in the fluid biosample, and the test result signal is dependent on the determined concentration value.

In accordance with an embodiment, the electronic circuit further comprises a wireless communication circuit for wirelessly transmitting the test result signal to at least one of a smart phone, tablet, computer, relay, access point and computer network.

FIG. 7 shows a system comprising a face mask 702, an EBC collector 704, a condensate forming surface 706, a fluid transfer 708, and a biosensor 710. A KN95 face mask is shown with a prototype retrofittable testing system including EBC collector having an aluminum foil condensate-forming surface, embossed fluid conductor channels, a microfluidic fluid transfer system and electronic biosensor electrodes.

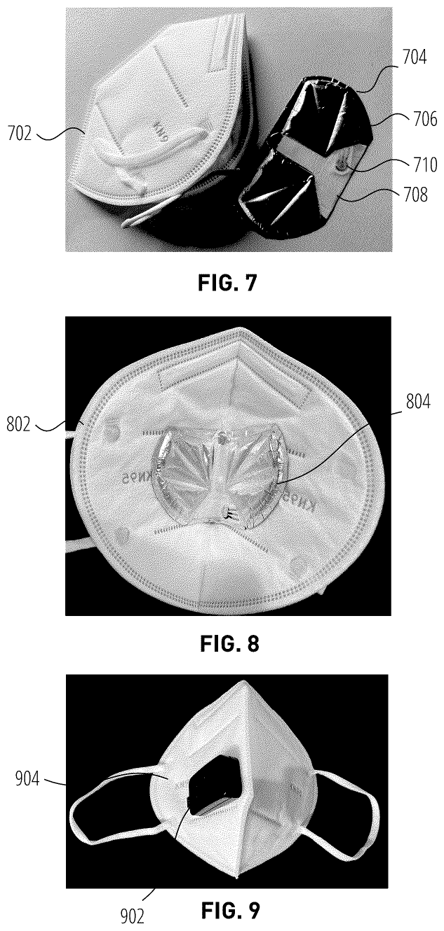

FIG. 8 shows a system comprising a face mask 802 and an EBC collector 804, showing the prototype testing system retrofit into the KN95 mask.

FIG. 9 shows a system comprising an electronics 902 and a face mask 904, showing the KN95 mask having removable testing and communication electronics disposed on the outside of the mask

FIG. 10 shows a system comprising a testing system support 1002, a biosensor 1004, an EBC collector 1006, a hydrophilic structure 1008, and a hydrophobic surface 1010. An Exhaled Breath Condensate Collector and a testing area is shown with ganged syndromic testing biosensors. In accordance with an exemplary prototype embodiment, a mask-based EBC collector is fabricated from 30 cm wide rolls of 120 μm thick natural virgin Polytetrafluoroethylene (PTFE) sheet (eplastics.com., USA), 10 cm wide 3M 465 double sided adhesive transfer tape (uline.com, USA) as well as a super absorbent polymer (SAP) powder, MediSAP 715 (M2 Polymer Technologies, Inc., Illinois, USA). A stamping jig can be constructed, for example, from 0.315 cm PTFE plate (eplastics.com, USA) and cut on a 100 W CO2 laser cutter (Orion Motor Tech, China). A Digital Combo Heat Press (Geo Knight, Massachusetts, USA) can be used to stamp the 127 μm thick PTFE sheet using the jig to form a pocket in the PTFE sheet for receiving a thermal mass mixture of water and the SAP. A second layer of the 127 μm PTFE sheet may be bonded to the heat stamped 127 μm PTFE sheet using a 3M 465 adhesive, sandwiching the thermal mass of water/SAP between layers of PTFE sheet. The completed PTFE/thermal mass/3M 465/PTFE laminated sandwich can be cut using a laser or die cutter into the final shape of the EBC collector that is configured and dimensioned to be inserted into a pre-existing face mask or built into a newly constructed face mask. The EBC collector constructed as described may be retro fit into various disposable face masks of different styles and constructions, including N95 and KN95 made by 3M and other manufacturers.

FIG. 11 shows a system comprising a pooling area 1102 and an adhesive 1104, showing the pooling area that forms a volume for holding the biosensors immersed in collected EBC.

FIG. 12 shows a system comprising an EBC collector 1202, a testing system support 1204, and a pooling area 1206. FIG. 12 shows an assembled EBC collector and pooling area. An exhaled breath condensate (EBC) collector converts breath vapor received from the lungs and airways of the test subject into a fluid biosample. The EBC collector includes a condensate-forming surface, and a thermal mass in thermal connection with the condensate-forming surface. A fluid transfer system transfers the EBC to at least one of a testing unit and an EBC containment vessel. The testing unit may be one or more electronic biosensors, one or more lateral flow assays or other microfluidic type testing systems, or a combination of the same. A testing system support for the EBC collector is configured and dimensioned to fit inside a face mask. The face mask forms an exhaled breath vapor containment volume to hold the exhaled breath vapor in proximity to the EBC collector to enable the exhaled breath vapor to efficiently coalesce into the fluid biosample.

As described in more detail herein, the testing unit may comprise a g-FET biosensor having a detection interface comprising a graphene layer functionalized with capture molecules, wherein the capture molecules are smaller than the Debye screening length. As an example of a syndromic testing device, the biosensors can be designed to bind to FluA, FluB, SARS N-protein (more conserved, slower to mutate protein across SARS viruses) and S-protein (faster to mutate, cause of the SARS-COV-2 variants).

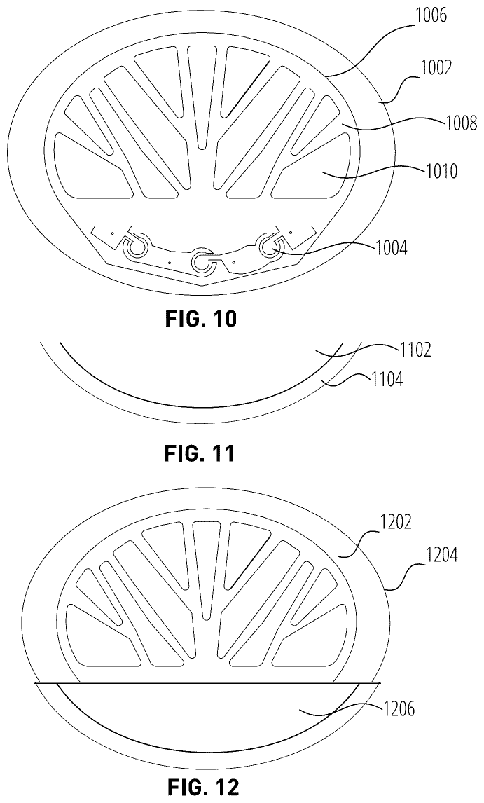

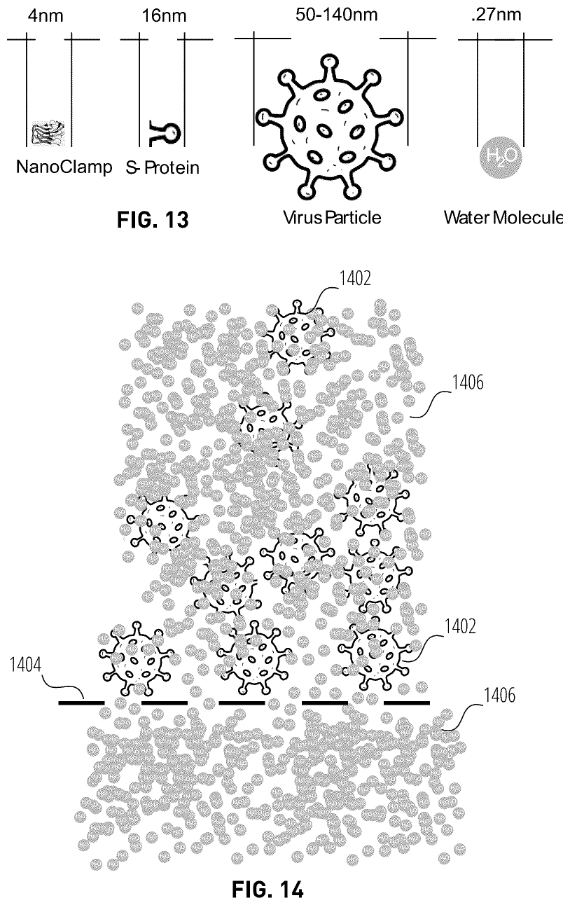

FIG. 13 shows the relative sizes of an engineered capture molecule known as a nanoCLAMP, an S-protein, a virus particle and a water molecule.

FIG. 14 shows an EBC concentrator having a semipermeable membrane for separating excess water from EBC to be tested with concentrated virus particles. The system includes a virus 1402, a selectively permeable membrane 1404, and a water 1406.

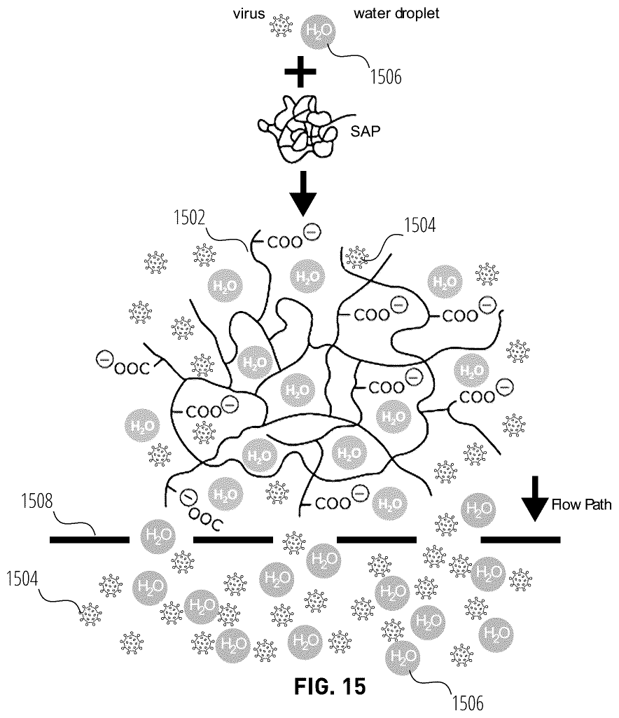

FIG. 15 shows the addition of a super absorbent polymer in the EBC concentrator. The system includes a SAP 1502, a virus 1504, a water 1506, and a selectively permeable membrane 1508.

nanoCLAMPs (nano-CLostridial Antibody Mimetic Proteins) are capture molecules developed by Nectagen (Kansas City, Kansas). Like aptamers and engineered antibodies, these capture molecules can be designed to bind to a specific antigen. nanoCLAMPS are based on an immunoglobulin-like, thermostable carbohydrate binding module from Clostridium hyaluronidase. nanoCLAMPs are small (4 nm×2.5 nm, 15 kDa) and have three variable loops comparable to immunoglobulin complementarity determining regions. nanoCLAMPS are within the Debye screening length and can be designed and enhanced to bind to different locations on the same antigen, different antigens, and large and small target molecules.

Particularly for larger macromolecules, experiments on capture molecules of different sizes indicates that the effectiveness of an FET-based sensor (where antigens are captured via specific binding to capture molecule-functionalized surfaces) is greatly affected by the Debye screening length. nanoCLAMPs typically bind selectively with nanomolar Kd's and release the captured antigen when treated with propylene glycol or glycerol.

For example, a nanoCLAMP can be designed to bind to biomarkers of viruses such as FluA HA, FluB NA, SARS N-protein (more conserved, slower to mutate protein across SARS viruses) and SARS S-protein (faster to mutate, a cause of the SARS-COV-2 variants). Thus, a multi-biomarker, syndromic testing system can be obtained through a ganged biosensor array that receives the collected EBC biosample. This ganged biosensor can be fabricated as an array of separate biosensor with printed electrodes and/or semi-conductor (e.g., g-FET) designs that are functionalized for a specific use-case through the selection of a respective capture molecule for a corresponding target molecule. In the case of a mask-based diagnostic platform, products made from the platform can share most if not all the components (mask, EBC collector, concentrator, fluid transfer, etc. Since the blood-air exchange that occurs in the lungs produces many biomarkers contained in the EBC, a specific use-case (e.g., tuberculous, lung cancer screening, environmental exposure, health and wellness, fitness, fatigue, vitamin deficiency, etc.) for the mask-based diagnostic system is determined by the addition of biosensors that are functionalized with the appropriate commercially available or custom designed capture molecule.

In accordance with an exemplary non-limiting embodiment, the biomarker concentrator can comprise a super absorbent polymer for preferentially absorbing water from the EBC into polymer chains of the super absorbent polymer. The target biomarker is not absorbed by the polymer chains and flows along with the EBC through the SAP and microfluidics structures of the diagnostic platform. As the EBC flows along through the SAP, the water content in the EBC is removed while the content of the target molecules remains constant, increasing the tested sample concentration of the target molecules.

A testing system includes an exhaled breath condensate (EBC) collector for converting breath vapor received from the lungs and airways of a test subject into a fluid biosample. The elements along the path can be tailored for conditioning the biosample before it is tested. For example, since EBC is mostly water, the target molecule concentration in the tested sample can be improved significantly by removing excess water. A biomarker concentrator concentrates a target biomarker portion in the fluid biosample to form a concentrated fluid biosample. Other test confounding constituents in the EBC can be removed from tested sample. For example, dissolved salts in the EBC can be removed via the SAP and semi/selectively permeable membrane actions described herein, or other constituent removal techniques such as precipitation reactions and filtering can be utilized. A biomarker testing unit receives the concentrated fluid biosample and tests the concentrated fluid biosample for the target biomarker.

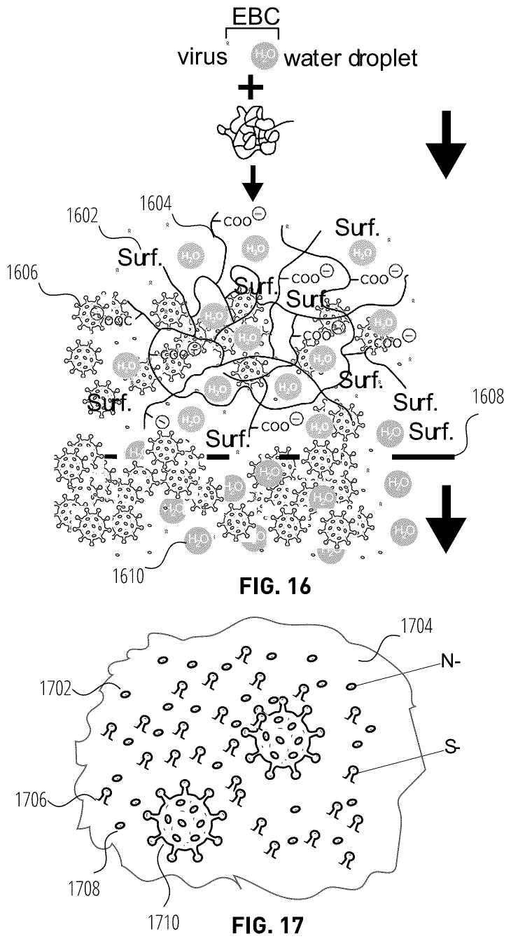

FIG. 16 shows the addition of a surfactant or lysing agent in the EBC concentrator. The system includes a surfactant 1602, a SAP 1604, a virus 1606, a selectively permeable membrane 1608, and a water 1610.

FIG. 17 shows an EBC tested sample having concentrated virus particles and lysed proteins. The system includes a fragment 1702, an exhaled breath vapor 1704, an S-protein 1706, an N-protein 1708, and a virus 1710.

A selectively permeable membrane is provided downstream in the flow path of the EBC sample from the super absorbent polymer. The membrane has a pore size that is configured and dimensioned to allow a portion of water in the EBC sample not absorbed in the super absorbent polymer blend, and the target analyte, to flow through the selectively permeable membrane. The pore size prevents the super absorbent polymer from flowing through the selectively permeable membrane resulting in a concentration of the target analyte in remaining water in the EBC sample on the permeate side of the membrane. The membrane allows virus particles and virus fragments to pass, along with the water not absorbed in the SAP to continue to flow towards the biosensor.

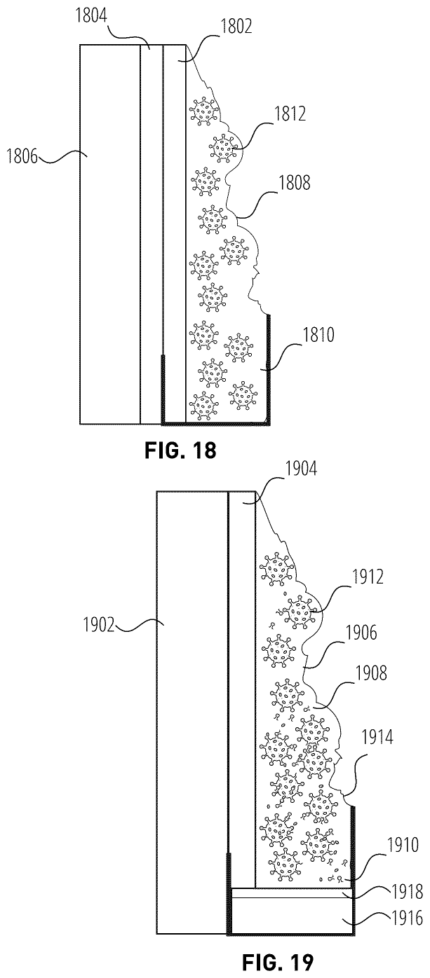

FIG. 18 is a cross section showing an EBC concentrator with a semipermeable membrane and wick disposed adjacent to a thermal mass. The system includes a selectively permeable membrane 1802, a wick 1804, a thermal mass 1806, an exhaled breath condensate 1808, a pooling area 1810, and a virus 1812.

The virus is concentrated in the EBC sample that accumulates at the pooling area formed by a barrier. Excess water in the EBC passes through the semipermeable membrane and is wicked away so that as the exhaled breath vapor is cooled into the EBC that forms on the EBC collector surface, excess water is continuously removed.

FIG. 19 is a cross section showing an EBC concentrator with a fluid conductor with lysing material and having a semipermeable membrane and wick in an EBC pooling area. The system includes a thermal mass 1902, a SAP 1904, an exhaled breath condensate 1906, an N-protein 1908, an S-protein 1910, a virus 1912, a virus-concentrated EBC 1914, a wick 1916, and a selectively permeable membrane 1918.

The biomarker concentrator may comprise a selectively permeable barrier for allowing excess water in the fluid biosample to pass through. The selectively permeable barrier blocks the target biomarker in the fluid biosample from passing through the selectively permeable barrier so that the fluid that is tested has a higher concentration of the target biomarker. An excess water absorbing wick can be provided for absorbing the excess water passing through the selectively permeable material.

Alternatively or in addition, a selectively permeable membrane can be provided upstream in the flow path of the EBC sample from the super absorbent polymer. The membrane has a pore size configured and dimensioned to allow a portion of water in the EBC sample to flow through the selectively permeable membrane to the super absorbent polymer. The pores prevent the target analyte from flowing through the membrane resulting in a concentration of the target analyte in remaining water in the EBC sample on the feed side of the membrane.

In accordance with an exemplary embodiment, an apparatus for testing exhaled breath condensate (EBC) for a target biomarker includes an EBC collector for converting breath vapor received from the lungs and airways of a test subject into an EBC biosample. The EBC biosample contains the target analyte. A biomarker concentrator comprises a super absorbent polymer layer in a flow path of the EBC biosample. During the collection, the EBC biosample sample is contacted with the super absorbent polymer layer and it absorbs a portion of water from the EBC biosample sample. The SAP layer does not absorb the target analyte, resulting in a concentration of the target analyte in remaining water in the EBC biosample. A biomarker testing unit receives the concentrated EBC biosample and tests the concentrated EBC biosample for a target biomarker.

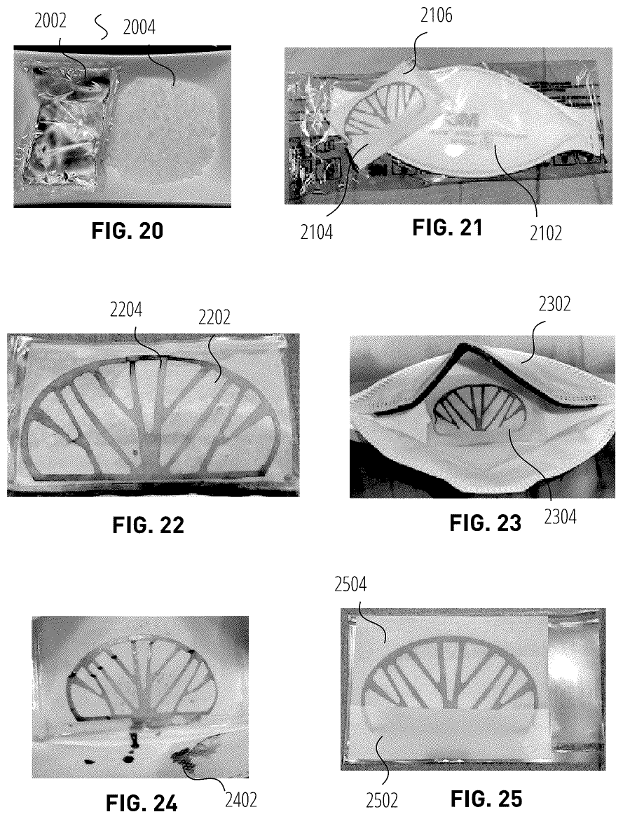

FIG. 20 shows the constituents of an endothermic reaction, including water and urea crystals. Other chemicals that react endothermically with water include ammonium nitrate and calcium ammonium nitrate. The system includes a water bag 2002, urea crystals 2004 which are examples of endothermic materials 2006.

FIG. 21 shows a retrofittable endothermic EBC collector and a pre-existing face mask. The system includes a face mask 2102, an EBC collector 2104, and a testing system support 2106.

FIG. 22 shows an endothermic EBC collector having hydrophilic channels on a hydrophobic field, with a dye incorporated in the hydrophilic channels to simulate a surfactant, precipitation reaction, and/or buffer additive. The system includes a hydrophobic surface 2202 and a hydrophilic structure 2204.

FIG. 23 shows an endothermic EBC collector retrofit into an existing face mask. The system includes a face mask 2302 and a testing system support 2304.

FIG. 24 shows a reacted endothermic EBC collector and dye colored collected EBC droplets formed from the exhaled breath condensate 2402.

In accordance with an exemplary embodiment, the EBC collector includes a condensate-forming surface and a thermal mass in thermal connection with the condensate-forming surface. The thermal mass may comprise at least a first chemical reagent and a second chemical reagent combinable to form an endothermic chemical reaction for absorbing thermal energy from the condensate-forming surface for converting the exhaled breath vapor to the EBC. FIG. 25 shows an endothermic EBC collector with a pooling area and dry buffer/surfactant. The system includes a pooling area 2502 and an EBC collector 2504.

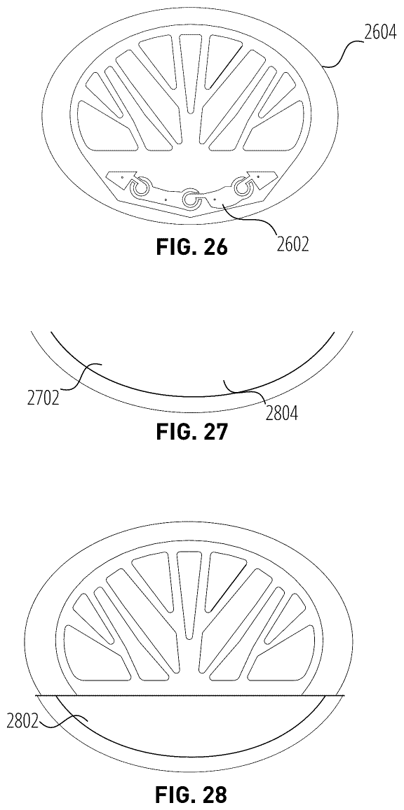

FIG. 26 shows an Exhaled Breath Condensate Collector and a testing area with ganged syndromic testing biosensors. The system includes a fragment 2602 and a testing system support 2604.

FIG. 27 shows a pooling area for immersing the biosensors in collected EBC, with dry buffer/surfactant in the pooling area. The system includes a pooling area 2702.

FIG. 28 shows an assembled EBC collector and pooling area with dry buffer/surfactant in the pooling area. The system includes a pooling area 2802 and a dry buffer/surfactant 2804.

The fluid transfer system may comprise at least one of a fluid conductor, a pooling area, and a microfluidics transfer path for controlling a flow of the fluid biosample received from the EBC collector. A target biomarker releasing material is disposed in or on said at least one of the fluid conductor, pooling area and microfluidics transfer path. The target biomarker releasing material may include at least one of a surfactant and a chemical lysing agent. The EBC collector can also include a target biomarker releasing structure for mechanically lysing at least one of a cell wall, encapsulating structure and viral envelope containing the target biomarker.

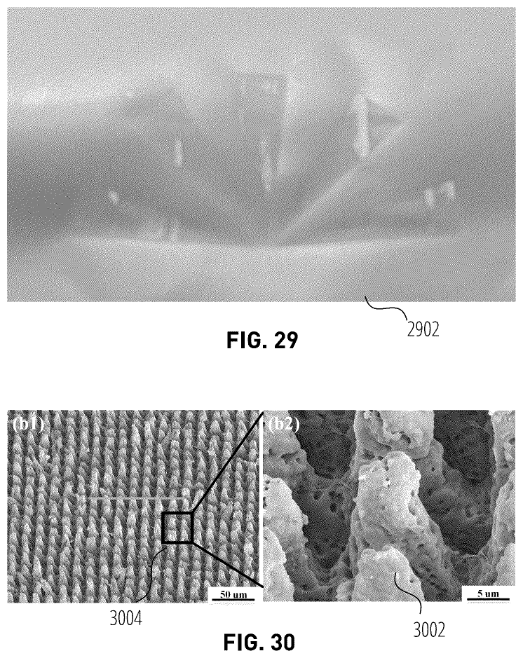

FIG. 29 shows a Teflon condensing surface with a textured fluid conductor. The system includes a patterned teflon sheet 2902.

FIG. 30 is a photomicrograph showing tunable water adhesion structures fabricated by laser ablation taken from a research paper, Superhydrophobic polytetrafluoroethylene surfaces with accurately and continuously tunable water adhesion fabricated by picosecond laser direct ablation, Qin et al., Materials and Design 173 (2019) 107782. The system includes teflon spikes 3002 and a patterned teflon sheet 3004.

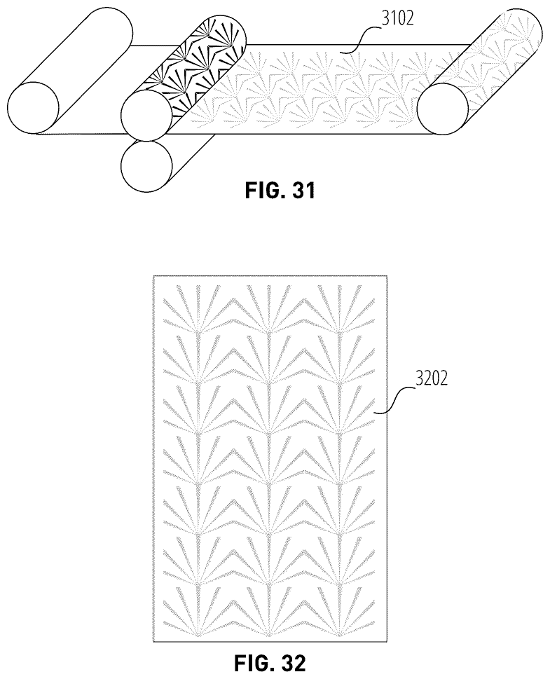

FIG. 31 shows a roll-to-roll process for mass producing a condensing surface with a textured fluid conductor. The system includes a patterned teflon sheet 3102.

FIG. 32 shows a sheet of condensing surfaces with textured fluid conductors. The system comprises a patterned teflon sheet 3202.

The sheet can be mass produced and be part of a materials stack for forming the EBC collector in a high volume manufacturing process, then the individual EBC collectors singulated from a finished roll of completed EBC collectors. The condensate-forming surface may have a relatively low energy surface property for limiting an adhesion of target biomarker to the condensate-forming surface. A fluid conductor disposed on the condensate-forming surface can be provided as a textured structure formed on the condensate-forming surface. The textured structure has a relatively higher energy surface property for guiding a flow of the EBC towards a desired direction.

A method for forming a condensate collector having fluid conductor channels on a substrate for guiding a flow of fluid towards a desired direction comprises providing the substrate having a surface having a relatively lower energy surface property. A textured structure forms fluid conductor channels on the surface having a relatively higher energy surface property for guiding a flow of fluid towards a desired direction.

The relatively lower energy surface property limits an adhesion of a target analyte on the surface and makes the surface relatively hydrophobic, and the higher energy surface property of the textured structure makes the channels relatively hydrophilic. The textured structure can be formed by at least one of laser ablation, sandblasting, etching and calendaring.

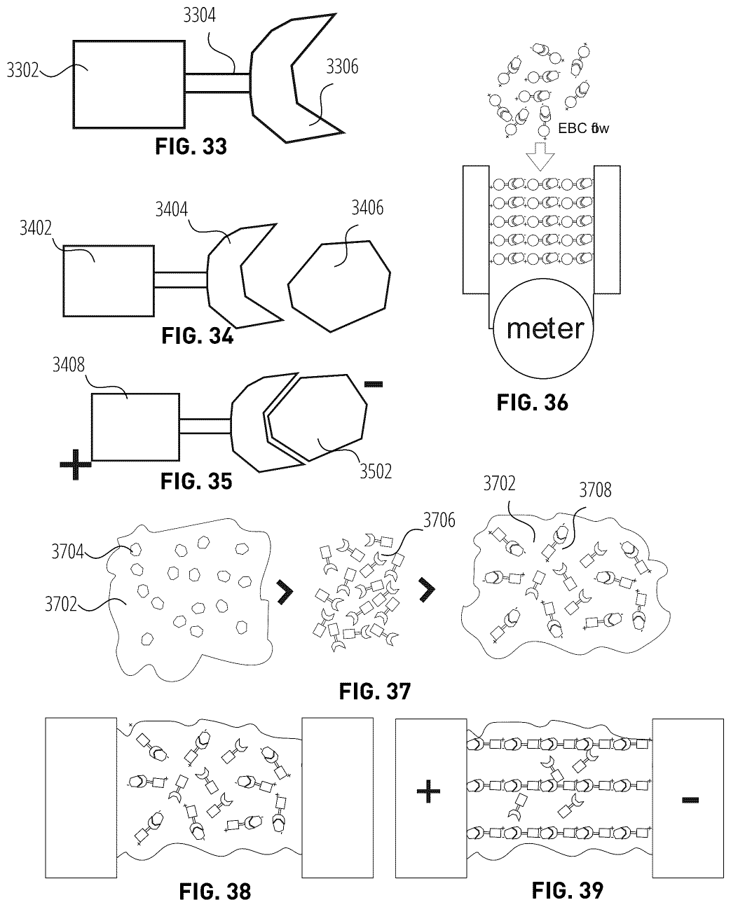

FIG. 33 shows a capture molecule structure having a label attached to a ligand. The system includes a label 3302, a linker 3304, and a capture molecule 3306.

FIG. 34 shows a capture molecule structure having a polarizable end and a ligand end for capturing a target analyte. The system includes a polarizable end 3402, a capture molecule 3404, a target molecule 3406, and a polarizable end 3408.

FIG. 35 shows the capture molecule structure with a captured target analyte forming a polar conjugate. The system includes a target molecule 3502.

FIG. 36 shows the alignment and testing of the polar conjugate to detect the target analyte in a fluid sample.

FIG. 37 shows a carrier fluid comprising a fluid sample with free floating target analyte molecules that is mixed with capture molecule structures to form free floating polar conjugates in the fluid sample. The system includes a carrier fluid 3702, a target molecule 3704, a capture molecule 3706, and a free-floating molecule 3708.

FIG. 38 shows the free-floating polar conjugates before aligning in an applied electric field.

A method for detecting a target analyte comprises providing a capture molecule structure having a ligand end and a polarizable end. When the capture molecule structure is disposed in a carrier fluid, the capture molecule structure is a free-floating element. A target analyte is provided as another free-floating element in the carrier fluid. The ligand end of the capture molecule structure binds to the target analyte and forms a free-floating polar conjugate having a positive end and a negative end. The polar conjugate is aligned in the carrier fluid in an electric field and an electrical property of the aligned polar conjugate is measured to detect the target analyte.

The carrier fluid can be a body fluid, including at least one of saliva, urine, exhaled breath condensate, blood and sweat. The step of measuring can involve pulsing the electric field for a duration and taking a measurement of the electrical property within a period of time after the duration, where the period of time is short enough to allow detecting the target analyte.

The carrier fluid can be a bio fluid sample and the capture molecule structure provided as a dry powder prior a step of mixing the capture molecule structure with the carrier fluid. The target analyte can be a constituent of the bio fluid sample. Alternatively, the carrier fluid can be an environmental fluid sample, and the target analyte is a constituent of the environmental fluid sample.

FIG. 39 shows the free-floating polar conjugates aligned in the applied electric field.

### Proof-of-Concept Experimental Results:

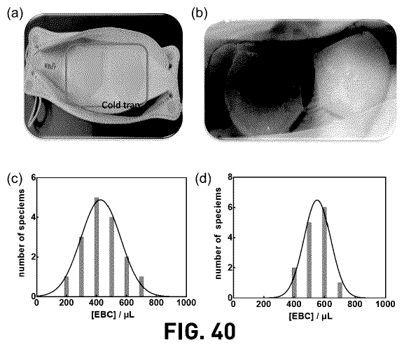

FIG. 40 shows (a) the inside of a proof-of-concept laboratory engineered mask showing an exhaled breath condensate (EBC) collector (cold trap) for converting breath vapor into a fluid sampling. The EBC collector is made of a Teflon-based condensate-forming surface, (b) an image of EBC formed on the Teflon collector after 5 min breathing into the mask, (c) a graph showing EBC volume collected in 5 minutes using the EBC Mask (n=14), and (d) a graph showing EBC volume collected in 5 minutes using a commercial RTube condensers (n=14).

The inventive Exhaled breath condensate (EBC) based diagnostic platform can be used for testing for SARS-COV-2 infectivity, and for other pathogens and environmental exposures. In proof-of-concept testing, a laboratory engineered mask allows collection of EBC by first cooling the mask for 30 min in the freezer, putting on the cooled mask and breathing into it for 5 min. EBC formed in the Teflon-lining of the inside of the masks is collected and directly deposited onto an electrochemical sensor modified with a SARS-COV-2 specific aptamer targeting the receptor-binding domain (RBD) region of the S1 spike protein as surface receptor. Using ferrocenemethanol as redox meditator before and after viral interaction allows discrimination between positive and negative EBC samples.

The mask-based EBC collection system is based on a commercial face mask fitted with an engineered EBC collector system based on a Teflon film cooling trap (a). To increase the EBC collection efficiency, the mask is placed into a freezer −20° C. for 30 min, before being placed over the mouth of the person to be tested. This polytetrafluoroethylene (PTFE) trap when cooled allows sample liquification on its surface, where the formed droplets can be collected with a pipette and used for analysis directly. The presence of a collection pool allows further collection of EBC (b) without the need for technical expertise. During the EBC collection the inside of the mask is not exposed to air and the risk of contamination of the EBC samples is negligible. Using this collection system, 400±150 μL of EBC can be collected within 5 minutes (c).

The collection efficiency was comparable to EBC collected by a commercially available RTube condenser (Respiratory Research Inc., USA) (d). The collection efficiency is person-dependent as seen in (c). However, in most cases the required 300 μL needed for further analysis was obtained in this manner. While indeed, collection of an equal volume of saliva is more efficient at a 5 min time span, saliva is a complex sample matrix containing proteases and other variable components that can impact most assays. This includes the potential degradation of the SARS-COV-2 S1 protein targeted by the aptamer employed in this test. The much cleaner EBC sample is therefore believed to be more suitable and reliable for rapid testing.

Various modifications and adaptations to the foregoing exemplary embodiments of this invention may become apparent to those skilled in the relevant arts in view of the foregoing description, when read in conjunction with the accompanying drawings. However, any and all modifications will still fall within the scope of the non-limiting and exemplary embodiments of this invention.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

1. An apparatus, comprising:
an exhaled breath condensate (EBC) collector for converting breath vapor received from the lungs and airways of a test subject into an EBC fluid biosample, the EBC collector including a cooling system for cooling a condensing surface wherein the breath vapor is coalesced into liquid droplets forming the EBC fluid sample;
a biomarker concentrator for concentrating a target biomarker portion in the fluid biosample to form a concentrated fluid biosample, wherein the biomarker concentrator comprises a selectively permeable barrier for allowing excess water in the fluid biosample to pass through the selectively permeable barrier and block the target biomarker in the fluid biosample from passing through the selectively permeable barrier;
a biomarker testing unit for receiving the concentrated fluid biosample and testing the concentrated fluid biosample for the target biomarker.

2. The apparatus according to claim 1, further comprising a testing system support for the EBC collector, wherein the testing system support is configured and dimensioned to fit inside a face mask, wherein the face mask forms an exhaled breath vapor containment volume to hold the exhaled breath vapor in proximity to the EBC collector to enable the exhaled breath vapor to coalesce into the fluid biosample.

3. The apparatus according to claim 1, further comprising an excess water absorbing wick for absorbing the excess water passing through the selectively permeable material.

4. The apparatus according to claim 1, further comprising applied-field-reactive capture molecule conjugate provided for capturing the target biomarker, the applied-field-reactive capture molecule conjugate having at least one applied-field-responsive end and a capture molecule end, wherein the capture molecule end binds to and captures the target biomarker.

5. The apparatus according to claim 4, further comprising a dissolvable adhesive for holding the applied-field-reactive capture molecule conjugate in a path of the fluid biosample, wherein water in the fluid biosample dissolves the dissolvable adhesive and allows the applied-field-reactive capture molecule conjugate to be free floating in the fluid biosample.

6. The apparatus of claim 5, wherein the applied-field-reactive capture molecule conjugate comprises a linker molecule disposed between the applied-field-responsive end and the capture molecule end, the linker molecule providing the applied-field-reactive capture molecule conjugate with electro-chemical properties wherein when the capture molecule end binds with the target biomarker at least one of a polarity and a conductivity of the applied-field-reactive capture molecule conjugate changes.

7. The apparatus according to claim 1, further comprising a droplet harvesting structure including a field for receiving the breath vapor and forming fluid droplets, and channels for receiving the fluid droplets from the field and channeling the fluid droplets.

8. The apparatus according to claim 1; wherein the biomarker concentrator further comprises a super absorbent polymer layer in a flow path of the EBC biosample where during the collection, the EBC biosample sample is contacted with the super absorbent polymer layer, where the super absorbent polymer absorbs a portion of water from the EBC biosample sample and does not absorb the target analyte resulting in a concentration of the target analyte in remaining water in the EBC biosample sample.

9. An apparatus for testing exhaled breath condensate (EBC) for a target biomarker, comprising:
an EBC collector for converting breath vapor received from the lungs and airways of a test subject into an EBC biosample, the EBC biosample containing the target biomarker, the EBC collector including a cooling system for cooling a condensing surface wherein the breath vapor is coalesced into liquid droplets forming the EBC fluid sample;
a biomarker concentrator comprising a super absorbent polymer layer in a flow path of the EBC biosample where during the collection, the EBC biosample sample is contacted with the super absorbent polymer layer, where the super absorbent polymer absorbs a portion of water from the EBC biosample sample and does not absorb the target analyte resulting in a concentration of the target analyte in remaining water in the EBC biosample sample; and
a biomarker testing unit for receiving the concentrated EBC biosample and testing the concentrated EBC biosample for a target biomarker.

10. The apparatus according to claim 9, wherein the biomarker concentrator further comprises a selectively permeable membrane having pores configured and dimensioned to allow the flow of the target biomarker through the selectively permeable membrane towards a testing unit.

11. The apparatus according to claim 9, further comprising a testing system support for the EBC collector, wherein the testing system support is configured and dimensioned to fit inside a face mask, wherein the face mask forms an exhaled breath vapor containment volume to hold the exhaled breath vapor in proximity to the EBC collector to enable the exhaled breath vapor to coalesce into the fluid biosample.

12. The apparatus according to claim 9, further comprising an excess water absorbing wick for absorbing the excess water passing through the selectively permeable material.

13. The apparatus according to claim 1, further comprising a droplet harvesting structure including a field for receiving the breath vapor and forming fluid droplets, and channels for receiving the fluid droplets from the field and channeling the fluid droplets.

14. The apparatus according to claim 1; wherein the cooling system comprises one of a thermal mass that is pre-cooled before use of the apparatus, freezable water/super absorbent polymer gel, a chilled metal plate and an endothermic thermal mass comprising two-part endothermic chemical system activatable just before use of the apparatus.

15. The apparatus according to claim 1; wherein the biomarker concentrator further comprises a super absorbent polymer layer in a flow path of the EBC biosample where during the collection, the EBC biosample sample is contacted with the super absorbent polymer layer, where the super absorbent polymer absorbs a portion of water from the EBC biosample sample and does not absorb the target analyte resulting in a concentration of the target analyte in remaining water in the EBC biosample sample.

16. An apparatus, comprising:
an exhaled breath condensate (EBC) collector for converting breath vapor received from the lungs and airways of a test subject into an EBC fluid biosample;
a biomarker concentrator for concentrating a target biomarker portion in the fluid biosample to form a concentrated fluid biosample, wherein the biomarker concentrator comprises at least one of a SAP for preferentially absorbing water from the EBC into polymer chains of the super absorbent polymer and a selectively permeable membrane having pores configured and dimensioned to allow the flow of the target biomarker through the selectively permeable membrane towards a testing unit, wherein as the EBC flows along through the SAP the water content in the EBC is removed while the content of the target molecules remains constant, increasing the tested sample concentration of the target molecules;
a biomarker testing unit for receiving the concentrated fluid biosample and testing the concentrated fluid biosample for a target biomarker.

17. The apparatus according to claim 16, wherein the biomarker concentrator further comprises a selectively permeable membrane having pores configured and dimensioned to allow the flow of the target biomarker through the selectively permeable membrane towards a testing unit.

18. The apparatus according to claim 16, further comprising a testing system support for the EBC collector, wherein the testing system support is configured and dimensioned to fit inside a face mask, wherein the face mask forms an exhaled breath vapor containment volume to hold the exhaled breath vapor in proximity to the EBC collector to enable the exhaled breath vapor to coalesce into the fluid biosample.

19. The apparatus according to claim 16, further comprising an excess water absorbing wick for absorbing the excess water passing through the selectively permeable material.

20. The apparatus according to claim 16, further comprising a droplet harvesting structure including a field for receiving the breath vapor and forming fluid droplets, and channels for receiving the fluid droplets from the field and channeling the fluid droplets.

21. The apparatus according to claim 16; wherein the biomarker concentrator further comprises a super absorbent polymer layer in a flow path of the EBC biosample where during the collection, the EBC biosample sample is contacted with the super absorbent polymer layer, where the super absorbent polymer absorbs a portion of water from the EBC biosample sample and does not absorb the target analyte resulting in a concentration of the target analyte in remaining water in the EBC biosample sample.

22-35. (canceled)
